(19) United States
(12) Patent Application Publication
(10) Pub. No.: US 2010/0280598 A1
(43) Pub. Date: Nov. 4, 2010

(54) VASCULAR GRAFT PROSTHESIS HAVING A REINFORCED MARGIN FOR ENHANCED ANASTOMOSIS

(75) Inventor: Peter Fox, Scottsdale, AZ (US)

Correspondence Address: C. R. Bard, Inc.;Bard Peripheral Vascular, Inc., 1415 W. 3rd Street, P.O. Box 1740, Tempe, AZ, 85280-1740, US

(73) Assignee: C.R. BARD, INC.

(21) Appl. No.: 12/810,822

(22) PCT Filed: Dec. 24, 2008
(86) PCT No.: PCT/US08/88312

§ 371 Date: Jun. 25, 2010

Related U.S. Application Data
(60) Provisional application No. 61/009,400, filed on Dec. 27, 2007.

(51) Int. Cl. A61F 2/06

(52) U.S. Cl. 623/1.32

(57) ABSTRACT

A vascular graft prosthesis having a reinforced margin designed and configured to strengthen the margin for various purposes, such as to minimize suture hole elongation and prevent suture line tearing during vessel anastomosis, thereby enhancing the vessel anastomosis and the anastomotic site.

### PRIORITY

This application claims the benefit of U.S. Provisional Patent Application No. 61/009,400, filed Dec. 27, 2007, which is incorporated by reference in its entirety into this application.

### FIELD

The present invention relates generally to vascular grafts or vascular graft prostheses, and more particularly to vascular graft prostheses such as those intended for use to alleviate or treat peripheral vascular disease (e.g., peripheral bypass grafts), as well as those intended for hemodialysis access (e.g., arteriovenous access grafts).

### BACKGROUND

Vascular grafts represent a very common class of biocompatible prosthetic implants used for a variety of purposes. For example, peripheral bypass grafts represent a specific type of vascular graft intended to treat peripheral artery occlusive disease (PAOD) (also known as peripheral vascular disease (PVD) and peripheral artery disease (PAD)), which describes the condition where the large peripheral arteries are stenosed or occluded. Peripheral bypass grafting is generally understood to describe the procedure in which an artificial vascular graft prosthesis is used to circumvent a stenosed or occluded area of the arterial vasculature. In another example, hemodialysis access grafts, or arteriovenous access grafts, comprise another specific type of vascular graft intended to provide hemodialysis “access” for patients suffering from renal disease, such as renal artery stenosis, or renal dysfunction or failure. An access graft is a subcutaneous device that is used to establish a fluid communication with, and to access, the patient's circulatory system. With an access graft, needles operable with a dialysis machine may be inserted into the graft to facilitate dialysis treatment.

Currently, there is a rapidly growing number of biocompatible materials, such as non-expanded or solid polytetrafluoroethylene (PTFE), expanded polytetrafluoroethylene (ePTFE) and solid fluorinated ethylene-propylene co-polymer (FEP), available for selection for use in a variety of medical/surgical applications. Naturally, some materials are better suited for particular applications than others. As a result, some of the primary concerns of surgeons, manufacturers and others are centered around the properties a certain material might exhibit in a given application, as well as the ability of that material to perform within what are often extremely sensitive biological settings. With respect to vascular graft prostheses, the type of biocompatible material selected for a particular application may depend upon, among other things, the ability of the material to reduce the potential for platelet adhesion or accumulation and fibrin deposition (which can lead to thrombosis), foreseeable patency rates, and the likelihood of the onset of intimal hyperplasia.

While the biocompatibility level of various materials is certainly a central concern, there are several other considerations with respect to a materials' ability to perform under given conditions, which may or may not be secondary to material biocompatibility. Specifically, with respect to vascular grafts, material properties such as tensile strength, suture tear resistance and suture hole elongation resistance of the material can all significantly affect the quality of the anastomosis. Tensile strength may be generally thought of as a measure of the maximum amount of tensile stress the material can be subjected to along a particular axis prior to failure or breakage. Suture line tear resistance may be generally thought of as a measure of how much force can be applied to a suture that has been sewn into the graft material before such force tears the graft material. Suture hole elongation resistance may be generally thought of as a measure of how much a suture hole stretches or elongates as pulled upon by a sewn suture subject to a tensile stress. One or all of these may be significant when considering the forces that will be acting on the vascular graft, the risk of suture hole bleeding during anastomosis and the potential for subsequent thrombosis, the time and care a surgeon must expend in order to establish a quality anastomosis, and whether or not a replacement graft may be necessary. This is particularly true proximate or about the terminal end and margin of the vascular graft where suturing occurs.

During anastomosis, for example venous anastomosis, a surgeon attempts to secure the terminal end of the graft (e.g., the cuffed venous end) to the wall of the portion of vein exposed following venotomy or at the venotomy site. Owing to often difficult operating conditions, haste, weak material or other factors present while performing the anastomosis, the surgeon will often experience tearing of the graft wall, and/or adverse elongation of the suture holes, thus increasing the risk of bleeding and subsequent thrombosis. Indeed, tensile forces applied to the graft by the surgeon while suturing, either intentional or inadvertent, can be too great, thus causing the graft to tear or suture holes to elongate. The size of the suture may also contribute to tearing, with thinner sutures exacerbating the problem. If tearing or elongation is egregious enough, such that excessive bleeding arises, failed anastomosis may be declared and a new graft required.

Another common problem is that suture hole elongation may increase the time to achieve anastomotic and suture hole hemostasis. This problem may prolong the time needed to complete the procedure by delaying the time before wound closure can be initiated and completed.

Most prior related vascular grafts are subject to these deficiencies due to the type of selected material and thin-wall makeup defining the tubular member and/or cuffed section, if present, of the vascular graft. Tensile strength and the potential for suture line tearing and/or suture hole elongation of vascular grafts are each particularly sensitive along a suture axis, which extends longitudinally along the suture between corresponding suture holes formed in the graft and vessel. While some materials are formed to inherently resist suture line tearing and elongation to some extent, such as a uniformly expanded PTFE material, this alone is typically not enough to withstand the forces acting on the vascular graft during typical anastomosis. In addition, a surgeon may place a vascular graft in a variety of different orientations during anastomosis, and there is no guarantee that sutures sewn through the graft material will necessarily be oriented in the direction of maximum suture tear resistance as provided by the material. There may be angulation of the sutures as the surgeon places each stitch, sometimes up to as much as 30 degrees.

### SUMMARY

In light of the problems and deficiencies inherent in the prior art, the present invention seeks to overcome these by providing a vascular graft prosthesis having a reinforced margin designed and configured to strengthen the margin for various purposes, such as to minimize suture hole elongation and prevent suture line tearing during vessel anastomosis, thereby enhancing the vessel anastomosis and the anastomotic site. The margin may be located at the terminal end of a cuffed or non-cuffed graft, and may be reinforced using integrally formed means for reinforcing (e.g., integral build-up at the margin of the same biocompatible material forming the tubular member and/or cuff) or separate means for reinforcing in the form of a reinforcing component (e.g., flex small beading or ribs, fittable sleeves, etc., that are disposed about and ultimately secured to the graft surface).

In accordance with one exemplary embodiment as embodied and broadly described herein, the present invention resides in a vascular graft prosthesis adapted for surgical anastomosis to a blood vessel, the vascular graft prosthesis comprising a generally tubular member defining a lumen for the passage of blood; an anastomotic component operable with the tubular member, and adapted for vessel anastomosis, the anastomotic component having a terminal end formation defining a margin; and means for reinforcing the anastomotic component to strengthen the margin, the means for reinforcing being adapted to enhance the vessel anastomosis and an anastomotic site during the vessel anastomosis.

The present invention also resides in a vascular graft prosthesis configured for surgical anastomosis to a blood vessel, the vascular graft prosthesis comprising a tubular member defining a lumen adapted for the passage of blood; and a terminal end formation of the tubular member adapted for surgical vessel anastomosis, the terminal end formation defining a margin, at least a portion of the terminal end portion having an increased wall thickness formed proximate the margin for strengthening the margin, the increased wall thickness enhancing the vessel anastomosis and an anastomotic site during the vessel anastomosis.

The present invention further resides in a vascular graft prosthesis configured for surgical anastomosis to a blood vessel, the vascular graft prosthesis comprising a tubular member defining a lumen adapted for the passage of blood, and formed of a first biocompatible material; a cuffed section extending from the tubular member adapted for surgical vessel anastomosis, the cuffed section comprising a margin; and a reinforcing component disposed at least partially about and secured to the cuffed portion, and adapted to strengthen the margin, the reinforcing component being formed of a second biocompatible material, the reinforcing component minimizing suture hole elongation and preventing suture line tearing of the terminal end formation during the vessel anastomosis.

The present invention still further resides in a method for reinforcing a vascular graft prosthesis to enhance an anastomotic site, the method comprising obtaining a vascular graft having a tubular member and an anastomotic component adapted for vessel anastomosis, the anastomotic component having a terminal end formation defining a margin; and reinforcing the margin of the anastomotic component to strengthen the margin, thus minimizing suture hole elongation and preventing suture line tearing of the anastomotic component during the vessel anastomosis.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 illustrates a perspective view of a vascular graft prosthesis in accordance with one exemplary embodiment of the present invention, wherein the vascular graft prosthesis comprises a cuff or cuffed section about one of its terminal ends to address and enhance the hemodynamics at the distal anastomosis;

FIG. 2-A illustrates a partial top view of the vascular graft prosthesis of FIG. 1;

FIG. 2-B illustrates a partial side view of the vascular graft prosthesis of FIG. 1;

FIG. 3-A illustrates a partial top view of a vascular graft prosthesis in accordance with another exemplary embodiment of the present invention, wherein the vascular graft prosthesis comprises a cuff or cuffed section of a different configuration about one of its terminal ends, also to address and enhance the hemodynamics at the distal anastomosis;

FIG. 3-B illustrates a partial side view of the vascular graft prosthesis of FIG. 3-A;

FIG. 4 illustrates a vascular graft prosthesis in accordance with still another exemplary embodiment of the present invention, wherein the vascular graft prosthesis comprises bifurcated flanges, or a bifurcated flanged cuff section to facilitate end to side anastomosis;

FIG. 5 illustrates a vascular graft prosthesis in accordance with still another exemplary embodiment of the present invention, wherein the means for reinforcing, or the reinforcing component, is disposed only partially about the cuff margin, namely along the sides, leaving the toe and heel sections devoid of reinforcement;

FIG. 6 illustrates a partial cross-sectional view of a cuffed section of an exemplary inventive vascular graft prosthesis, wherein the cuffed section comprises an exterior wall surface having flex small beading disposed thereabout proximate the terminal end formation to strengthen the margin, and wherein the beading comprises a semi-circular or curved profile or cross-section;

FIG. 7 illustrates a partial cross-sectional view of a cuffed section of an exemplary inventive vascular graft prosthesis, wherein the cuffed section comprises an exterior wall surface having flex small beading disposed thereabout proximate the terminal end formation to strengthen the margin, and wherein the beading comprises a rib having a rectangular or linear profile or cross-section;

FIG. 8 illustrates a partial cross-sectional view of a cuffed section of an exemplary inventive vascular graft prosthesis, wherein the cuffed section comprises a non-uniform wall thickness with excess material built-up about the terminal end formation to strengthen the margin, and wherein the built-up portion is formed of the same biocompatible material as the cuffed section;

FIG. 9 illustrates a vascular graft prosthesis in accordance with still another embodiment of the present invention, wherein the vascular graft prosthesis is capable of receiving and having secured thereto, one of several differently configured sleeves, each of which conform substantially to at least a portion of a cuffed section of the vascular graft prosthesis to strengthen a margin of the cuffed section; and

FIG. 10 illustrates a detailed, partial perspective view of an exemplary inventive vascular graft prosthesis shown partially anastomosed to a section of the peripheral vasculature, namely to a section of vein.

### DETAILED DESCRIPTION OF EXEMPLARY EMBODIMENTS

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

The term “anastomotic component,” as used herein, shall be understood to mean the one or more components of a vascular graft that are physically anastomosed to a vessel. Exemplary anastomotic components include, but are not limited to, venous or arterial terminal end portions of a tubular member of a vascular graft having or defining a margin, venous or arterial terminal end portions of a tubular member of a vascular graft having a single or multiple-bulb cuff or flange configuration defining a margin, annular or other exterior flanges located between terminal ends and about the tubular member of a vascular graft and that define a margin.

The present invention describes a method and system for reinforcing the margin of an anastomotic component of a vascular graft to enhance anastomosis and minimize suture hole elongation and suture line tearing. Reinforcement is effectively carried out by increasing the wall thickness at the margin, which can be accomplished using any one of a variety of means, as will be discussed below. Although it is contemplated that grafts of different types may benefit from the present invention, those formed from synthetic materials such as PTFE and FEP are primarily discussed below as these currently represent the most predominant graft types.

The present invention technology of reinforcing the anastomotic component (e.g., the perimeter edge or margin of a terminal end formation (cuffed or non-cuffed) integral with a tubular member) of a vascular graft prosthesis provides several significant advantages over prior related vascular graft prostheses devoid of margin reinforcement, some of which are recited here and throughout the following more detailed description. First, the risk of suture line tearing and suture hole elongation, such as during the performance of venous or arterial anastomosis, is at least reduced, if not eliminated, either of which can increase the risk of suture hole bleeding that may induce thrombosis within the vessel potentially causing vascular occlusion. Second, integrity of the anastomotic component is preserved. For example, integrity of the margin of a cuff (the anastomotic component) of a vascular peripheral bypass graft prosthesis is maintained as the reinforcing means or reinforcing component (e.g., flex small beading, fittable sleeve, etc.) located or disposed about all or part of the margin functions to strengthen and fortify the margin, thus decreasing the potential for tearing. Third, the presence of the reinforcing means or component discourages practitioners (e.g., surgeons) from trimming the anastomotic component that could adversely affect the hemodynamic and clinical performance of the vascular graft prosthesis. However, it is recognized that the addition of the reinforcing means should not compromise the practitioners ability to properly perform the anastomosis. Fourth, reinforcement of the anastomotic component may be localized in select regions or areas particularly prone to suture line tearing and/or hole elongation, or continuously applied in an uninterrupted manner, such as circumferentially about the margin. Fifth, anastomosis is enhanced in terms of efficiency and quality as the factors causing a reduction in each of these, namely line tearing and hole elongation, are eliminated or at least significantly minimized. Sixth, the time to suture hole hemostasis is significantly reduced as suture hole elongation is minimized.

Each of the above-recited advantages will be apparent in light of the detailed description set forth below, with reference to the accompanying drawings. These advantages are not meant to be limiting in any way. Indeed, one skilled in the art will appreciate that other advantages may be realized, other than those specifically recited herein, upon practicing the present invention.

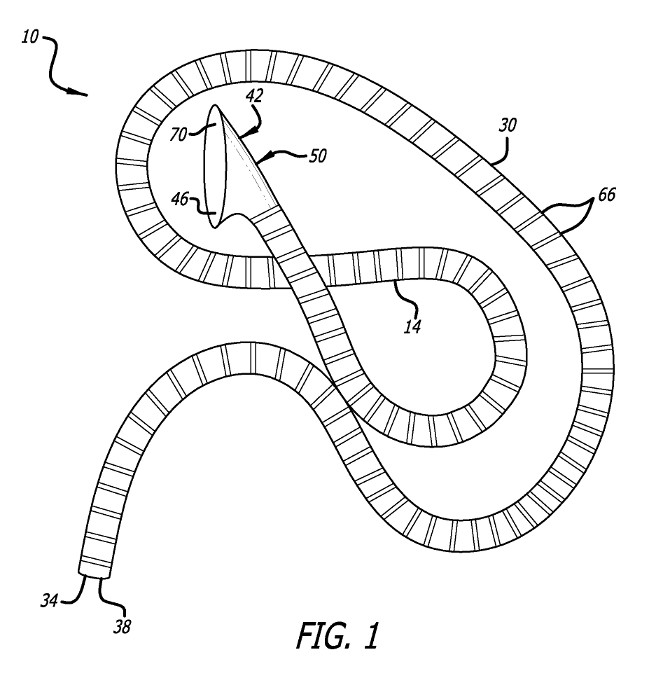

With reference to FIGS. 1-3, illustrated is a vascular graft formed in accordance with one exemplary embodiment of the present invention. As shown, the vascular graft 10 resides in the form of a peripheral bypass graft, such as is used to treat peripheral vascular disease (PAD). The vascular graft 10 comprises an elongate tubular member 14 that defines a lumen to facilitate the passage of fluids, such as blood, and to permit bypass of an occluded or diseased vessel. The tubular member 14 comprises a generally tubular shape, typically having a circular cross-section and a thin wall design. However, the tubular member 14 may comprise a variety of different sizes and configurations.

The vascular graft further comprises one or more anastomotic components used to facilitate vessel anastomosis. In the embodiment shown, located at one end of the tubular member 14 is a proximal arterial end 34 adapted for proximal arterial anastomosis. The proximal arterial end 34 comprises a terminal end formation, being essentially an end of the tubular member 14 defining a perimeter or margin 38 located about the most distal portion of the proximal arterial end 34 (rather than an end formed by a portion of cuff material doubled back on itself to create a fold). Opposite the proximal arterial end 34 of the tubular member 14 is a distal arterial end 42 having another anastomotic component, also adapted for arterial anastomosis. In the particular embodiment shown, the anastomotic component at the distal arterial end 42 comprises an outwardly flared cuff 50 extending from the tubular member 14 in an integral and continuous manner, which cuff 50 comprises a terminal end formation defining a perimeter or margin 46 located about the most distal portion of the cuff 50 at the distal arterial end 42 (rather than an end formed by a portion of cuff material doubled back on itself to create a fold). The cuff 50 comprises a toe section 54 projecting away from the tubular member 14 in one direction, and a heel section 58 projecting away from the tubular member 14 in another direction.

The tubular member 14 and/or cuff 50 may be formed from a suitable biocompatible material such as, for example, from polytetrafluoroethylene, polyester, polyurethane, or fluoropolymers, such as perfluoroelastomers, and combinations thereof. However, in the embodiment shown, the cuff 50 and tubular member 14 are each formed of expanded polytetrafluoroethylene (ePTFE).

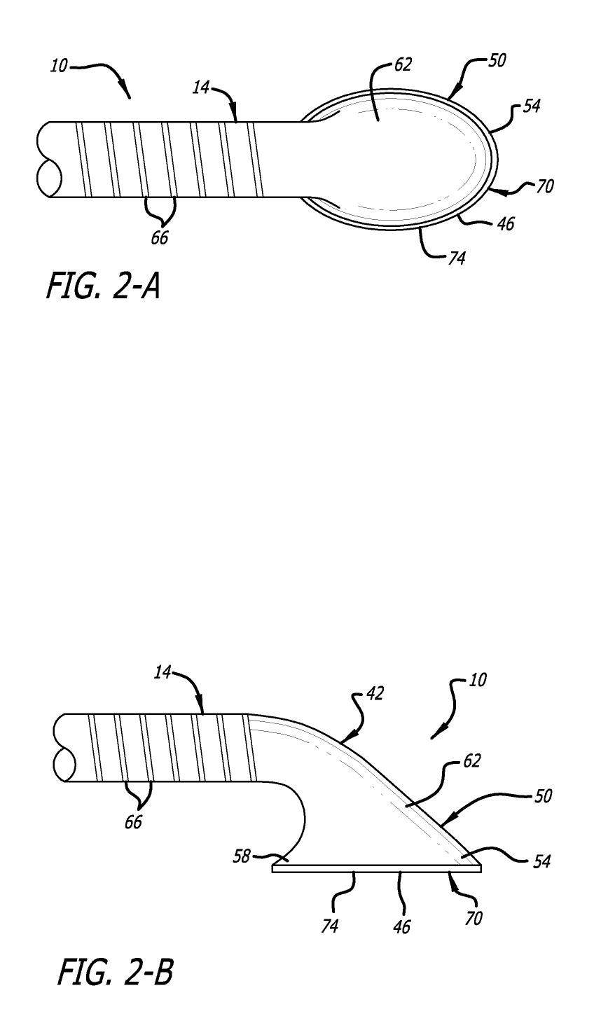

In many cases, the cuff 50 will comprise a thinner wall than the tubular member 14. This is by virtue of the current manufacturing process whereby the cuff is fashioned by expanding it radially over a mandrel or mold. This results in a distal cuff margin that is thinner than the wall of the tubular member and other portions of the graft. In addition, depending upon the application, the tubular member 14 may lack sufficient diametric mechanical rigidity. As such, as is the case with the exemplary vascular graft 10 of FIGS. 1-2-B, the vascular graft 10 may further comprise an external reinforcement, such as beading 66 of biocompatible material helically and circumferentially wound or disposed about the exterior surface 30 of the tubular member 14, which beading 66 is similar to the flex small beading found on various prior related ePTFE vascular grafts, such as the Dynaflo® and Distaflo® vascular grafts of Bard Peripheral Vascular, Inc. (a division of C. R. Bard, Inc.). The beading 66 functions, among other things, to reduce kinking, provide crush resistance and provide other advantages as commonly known in the art. The beading 66 may comprise various types of biocompatible materials, but typically comprises non-expanded or solid polytetrafluoroethylene (PTFE), or solid fluorinated ethylene-propylene co-polymer (FEP). Non-expanded or solid PTFE is significantly more rigid than expanded polytetrafluoroethylene (ePTFE) material due to its higher density and absence of interstitial voids. The beading can be impregnated with a radiopaque material, such as barium sulfate or hydroxyapatite, to increase visibility of the vascular graft 10 under radio imaging (e.g., x-ray).

Unlike prior related vascular grafts, the present invention vascular graft 10 further comprises means for reinforcing an anastomotic component, and particularly the margin of the anastomotic component, of a vascular graft, wherein the anastomotic component may or may not be located about a terminal end (e.g., such as is the case with grafts having cuffs located and extending circumferentially about the exterior of the tubular member). As discussed above, there are several problems associated with prior related grafts resulting in less than optimal vessel anastomosis. Specifically, during the actual anastomosis procedure, suture tensile forces acting within sutures and on the vascular graft caused by the surgeon pulling and tightening the sutures, as well as manipulating the vascular graft into a desired position, can create ominous conditions leading to bleeding and possibly ultimate thrombosis, which in such case, the thrombus must be removed and/or a new graft implanted. For example, if suture tensile forces are too great, suture line tearing can occur, which results in a suture completely tearing from the anastomotic component of the vascular graft. In this case, measures must be taken to correct the tear and maintain the integrity of the suturing, or a new graft obtained and the anastomosis repeated. Even if suture tensile forces are not strong enough to induce tearing within the anastomotic component, they can still place significant strain on the anastomotic component to the point of suture hole elongation. If the elongation is considerable, again bleeding and possibly ultimate thrombosis can occur leaving the patient in the same ominous condition.

Addressing the problems of suture line tearing and suture hole elongation, the present invention sets forth diverse means for reinforcing an anastomotic component in order to strengthen the margin of the anastomotic component, thus reducing or eliminating the risk of suture line tearing and suture hole elongation during anastomosis. In one exemplary embodiment, means for reinforcing the anastomotic component may comprise a separate reinforcing structure or component 70 disposed about and secured to at least a portion of the exterior surface 62 of the cuff 50 immediately proximate or adjacent the margin 46, thus permitting the reinforcing structure 70 to actually help define the margin boundary. As shown in FIGS. 1-2B, the reinforcing component 70 comprises a band of beading 74, such as flex small beading, similar in size, geometry and material makeup as the helical beading 66 disposed about the tubular member 14. The cross-sectional area of the reinforcing beading 74 may be smaller, larger or the same as that of the helical beading 66.

The reinforcing flex small beading 74 may be secured to the exterior surface 62 of the anastomotic component or cuff 50 using means and materials known in the art. In one embodiment, the reinforcing beading 74 may be disposed about and sintered to the cuff 50 proximate the margin 46 prior to laser trimming of the vascular graft. Other securing means may be used to secure the beading 74 to the cuff 50, including, but not limited to, biocompatible adhesives, mechanical fasteners or means (e.g., fasteners, staples, sutures, etc.), and any others recognized by those skilled in the art and their combinations. The same may be said for all of the exemplary embodied means for reinforcing discussed herein.

The means for reinforcing may comprise various types of biocompatible materials similar to those discussed elsewhere herein. For example, reinforcing means in the form of beading may be formed of a non-expanded or solid PTFE, or a solid FEP co-polymer material similar to the helical beading described above. It is to be noted that the means for reinforcing may be comprised of the same material or different material as the tubular member and/or the anastomotic component. Similarly, the means for reinforcing may be impregnated with a radiopaque material, such as barium sulfate or hydroxyapatite, to increase visibility of the vascular graft under radio imaging (e.g., x-ray), which can be used to provide evidence as to whether a surgeon trimmed the anastomotic component. It is also noted herein that the anastomotic component of the present invention vascular graft is not intended to be trimmed as this would disrupt or remove the reinforcing means defeating its purpose. In addition, trimming the vascular graft to remove or interfere with the reinforcing means may adversely affect the hemodynamic and clinical performance of the vascular graft.

Generally speaking, the vascular graft prosthesis, including the means for reinforcing the margin of the anastomotic component, as deployed within the body, may be formed from one or more materials having a suitable degree of biocompatibility. These biocompatible materials, or biomaterials, are generally described as materials, natural or man-made synthetic, that comprise whole or part of a living structure or biomedical device intended to replace part of a living system or to function in intimate contact with living tissue. Biocompatible materials are intended to interface with biological systems to evaluate, treat, augment, perform or replace any tissue, organ or function of the body. Exemplary biocompatible materials include, but are in no way limited to, polytetrafluoroethylene (PTFE), expanded polytetrafluoroethylene (ePTFE), polyester, polyurethane, or fluoropolymers, such as perfluoroelastomers, and combinations thereof. It will be apparent to those skilled in the art that other biocompatible materials may exist that may be used, or that others being developed may also be used. As the focus of the present invention is not, per se, on the type of material used to form the one or more components of the vascular graft prosthesis, it is intended to be understood that other suitable biocompatible materials not mentioned herein are contemplated for use.

While several suitable biocompatible materials exist in the art, the use of expanded polytetrafluoroethylene (ePTFE) as a nonviable, bio-inert barrier material is well known, and is a popular material selection for many graft prostheses. For example, the tubular member, the cuffed section, and the means for reinforcing of the present invention vascular graft prosthesis may be formed from ePTFE, PTFE, or a combination of these. As discussed herein, a tubular member and a cuffed section may be formed of ePTFE, with means for reinforcing being formed from PTFE or some other or suitable biocompatible material. Depending upon the particular application, ePTFE may provide one or more advantages over other materials with respect to the tubular member and anastomotic components, with solid PTFE providing one or more advantages over other materials with respect to means for reinforcing. However, the type of reinforcing means desired, the intended application of the vascular graft, and other factors may dictate the type of materials used for each component part.

It is also contemplated that one or more bioactive agents may be incorporated into the components of the vascular graft prosthesis, including the tubular member 14, the cuff 50 and/or the means for reinforcing. Exemplary bioactive agents include, but are not limited to, activated charcoal, carbon particles, graphite particles, vasodilator, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II.sub.b/III.sub.a inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine cladribine); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

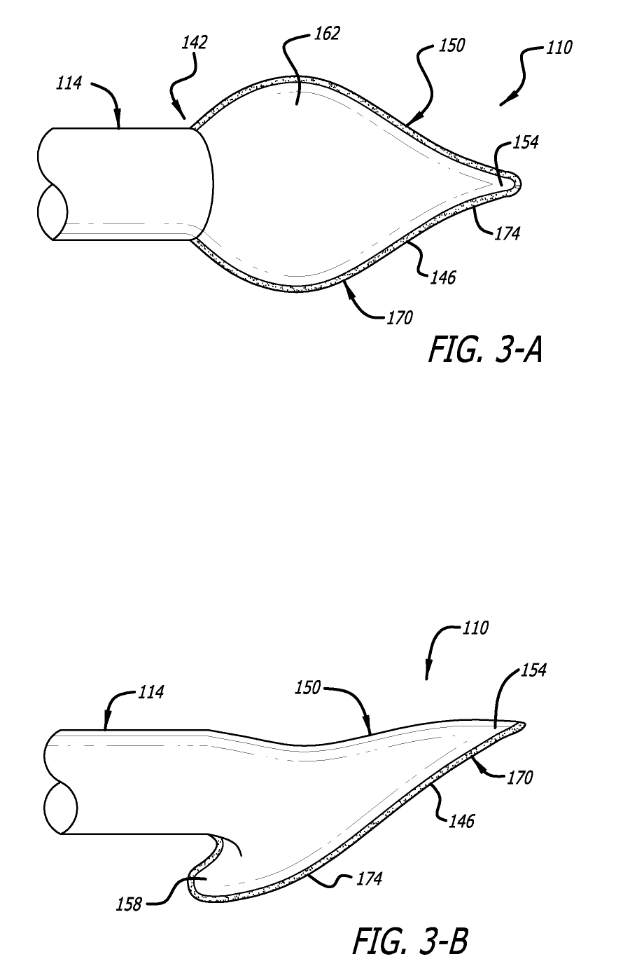

Referring now to FIGS. 3-A and 3-B, shown is a vascular graft prosthesis 110 formed in accordance with another exemplary embodiment of the present invention. As shown, the vascular graft 110 resides in the form of a hemodialysis access graft used to provide efficient access by a needle for patients undergoing dialysis. In this particular embodiment, the vascular graft 110 comprises a tubular member 114 and an anastomotic component at the venous end 142 in the form of an outwardly flared cuff 150 extending from the tubular member 114 in an integral and continuous manner, which cuff 150 comprises a terminal end formation defining a perimeter or margin 146 located about the most distal portion of the cuff 150. The cuff 150 comprises a toe section 154 projecting away from the tubular member 114 in one direction, and a heel section 158 projecting away from the tubular member 114 in another direction. Different from the vascular graft 10 of FIGS. 1, 2-A and 2-B, the cuff 150 of the vascular graft 110 of FIGS. 3-A and 3-B is specifically designed to improve patency by optimizing the hemodynamics at the venous anastomosis. The configuration of the cuff is similar to that found on the vascular graft manufactured and sold by Bard Peripheral Vascular, Inc. (a division of C.R. Bard, Inc.) under the trademark Venaflo®.

Similar to the vascular graft 10 of FIGS. 1, 2-A and 2-B, the vascular graft 110 of FIGS. 3-A and 3-B further comprises means for reinforcing the anastomotic component or cuff section 150 in the form of a separate reinforcing structure or component disposed about and secured to at least a portion of the exterior surface 162 of the cuffed section 150 immediately proximate or adjacent the margin 146, thus permitting the reinforcing structure to actually help define the margin boundary. As shown, the reinforcing component 170 comprises a band of beading 174, such as flex small beading. The function, makeup and characteristics of the beading 174 are similar to those described above with respect to the vascular graft 10 of FIGS. 1, 2-A and 2-B, which subject matter is incorporated herein. Of course, means for reinforcing the anastomotic component may include other structures as described herein, or that would be obvious to one skilled in the art.

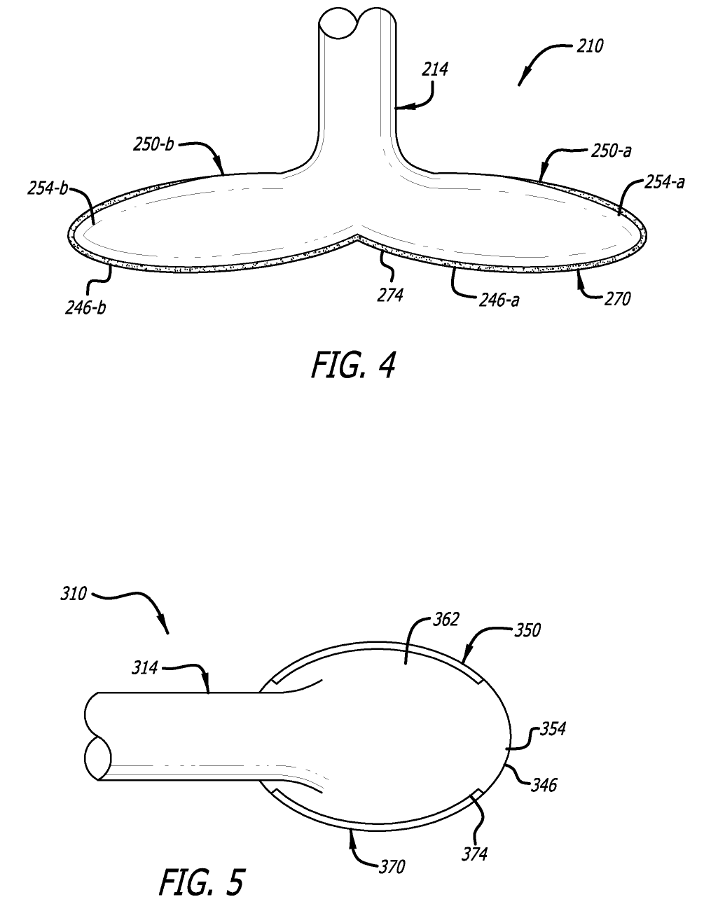

FIG. 4 illustrates another vascular graft prosthesis in accordance with still another exemplary embodiment of the present invention. In this embodiment, the vascular graft prosthesis 210 resides in a form particularly suited or adapted for end-to-side anastomosis to facilitate femoro-crural or below the knee bypass. As shown, the vascular graft 210 comprises a tubular member 214 and a flared, double-bulb cuff configuration, shown as cuffs 250-a and 250-b, projecting away from the tubular member 114 in different directions. Each cuffed section 250-a and 250-b comprises a toe section, shown as toe sections 254-a and 254-b, respectively. In addition, each cuffed section 250-a and 250-b comprises a terminal end formation defining margins 246-a and 246-b, respectively.

The vascular graft 210 further comprises means for reinforcing the anastomotic component or cuffed sections 250-a and 250-b. As shown in this particular embodiment, means for reinforcing comprises a separate reinforcing structure or component 270 disposed about and secured to at least a portion of the exterior surfaces of each cuffed section 250-a and 250-b, proximate the respective margins 246-a and 246-b. As shown, the reinforcing component comprises a band of beading 274, such as flex small beading. The function, makeup and characteristics of the beading 274 are similar to those described above and shown in FIGS. 1-3-B, which subject matter is incorporated herein. Of course, means for reinforcing the anastomotic component may include other structures as described herein, or that would be obvious to one skilled in the art.

FIG. 5 illustrates an exemplary vascular graft prosthesis 310 formed similar to the one described above and shown in FIGS. 1, 2-A and 2-B. However, the graft 310 may also be formed to comprise other known configurations, such as those illustrated in FIGS. 3-A and 3-B, or that illustrated in FIG. 4, or others known in the art. Unlike the grafts described above, graft 310 comprises an anastomotic component in the form of a cuffed section 350 extending from a tubular member 314, wherein the anastomotic component is reinforced with means for reinforcing in the form of a separate reinforcement component 370 that extends only partially about the margin 346. More particularly, the cuffed section 350 comprises a toe section 354 and heel section (not shown) that are devoid of reinforcement. As shown, reinforcement beading 374 is disposed about and secured to the exterior surface 362, but is selectively located only about the sides of the cuffed section 350, without extending to or about the toe or heel sections. This particular embodiment illustrates the idea that only select portions of the margin may be reinforced and strengthened. The location of the beading 374 about the sides is not intended to be limiting in any way. Indeed, any part of the anastomotic component and corresponding margin may be reinforced, including selectively reinforcing only the toe and/or heel sections if so desired.

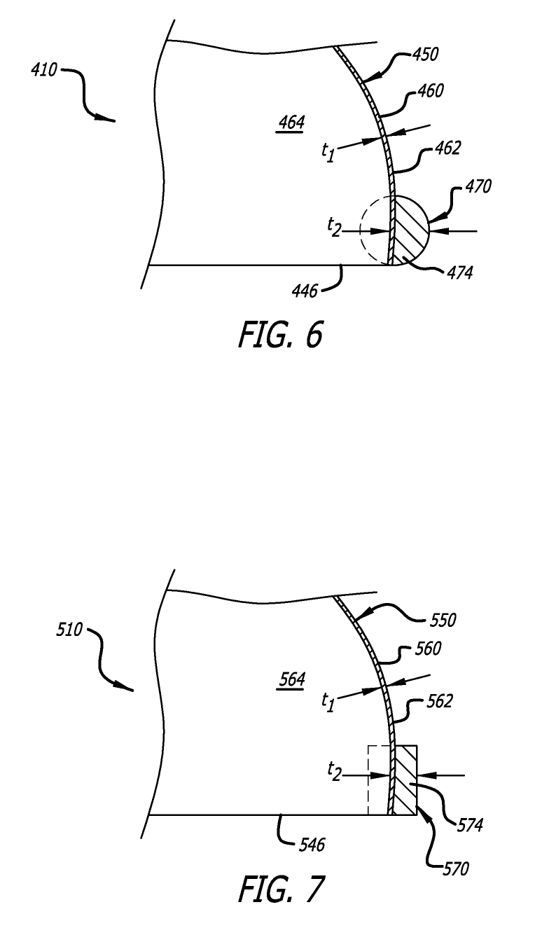

Means for reinforcing the anastomotic component has the advantageous effect of increasing the wall thickness of the anastomotic component at least proximate the margin and perhaps all over depending upon the configuration of the means for reinforcing. An increased wall thickness functions to strengthen the margin, and overall enhance the vessel anastomosis and the anastomotic site of the vessel anastomosis. As shown in partial cross-section in FIG. 6, an exemplary vascular graft 410 comprises an anastomotic component in the form of a cuffed section 450 having a wall 460 defining exterior and interior surfaces 462 and 464, respectively. The wall 460 is shown as comprising a thickness t₁ that may or may not be the same thickness as the wall structure of the tubular member (not shown). Disposed proximate the margin 446 and secured to the exterior surface 462 of the wall 460 of the cuffed section 450 is means for reinforcing the anastomotic component in the form of a separate reinforcing component 470 or beading 474 similar to that described above, and having a semi-circular or curved cross-section. The beading 474 is located so as to effectively help define the margin 446, and particularly a thickness of the margin 446. The beading 474 is shown as comprising a thickness t₂, so as to effectively increase the thickness of the wall 460 at the margin, where the total thickness is established by the addition of the beading 474 to the wall 460, or t₁+t₂. The beading 474 may be made of any biocompatible material, as discussed herein. As shown, the beading 474 is formed of a solid PTFE material, with the cuffed section 450 being formed of ePTFE material. FIG. 6 also shows that means for reinforcing, such as flex small beading, may be applied or disposed and secured to the wall 460 about the interior surface 464 of the cuffed section 450, either in place of or to complement the beading on the exterior surface 462.

FIG. 7 shows a partial cross-section of an exemplary vascular graft 510 similar to the vascular graft 410 of FIG. 6. However, in this particular embodiment, the cuffed section 550 has disposed about and secured to its exterior surface 562, at the margin 546, means for reinforcing 30 the anastomotic component (e.g., cuffed section 550), shown as a reinforcing component 570 in the form of a rib 574 having a rectangular or linear side cross-section. The rib 574 may be configured to function in the same manner as the beading described above, comprising a thickness t₂ that effectively increases the thickness t₁ of the wall 560 at or proximate the margin 546 (the total thickness being t₁+t₂). Again, as shown, a second rib may be disposed about and secured to the interior surface 564 of the cuffed section 550 if desired.

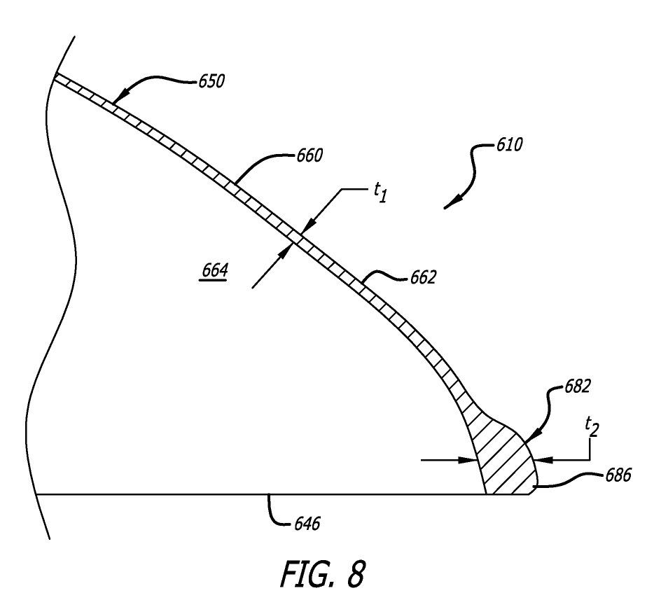

With reference to FIG. 8, illustrated is an exemplary vascular graft 610 comprising an anastomotic component in the form of a cuffed section 650 having a wall 660 defining exterior and interior surfaces 662 and 664, respectively. The wall 660 is shown as primarily comprising a thickness t₁ that may or may not be the same thickness as the wall structure of the tubular member (not shown). Located at or proximate the margin 646 is means for reinforcing the anastomotic component, which means is integrally formed with, or is an extension of, the wall 660 of the cuffed section 650. Specifically, means for reinforcing is shown as comprising an integral reinforcing component 682 comprising a built-up region 686 of material, having a thickness t₂ at the thickest portion, that extends around the margin 646 of the cuffed section 650 so as to provide a non-uniform wall thickness of the cuffed section 650. The built-up region 686 effectively increases the thickness of the margin 646 so as to strengthen the margin 646 for enhanced anastomosis. The built-up region 686 functions to reinforce the margin 646 to minimize suture line tearing and suture hole elongation similar to the separate reinforcing components discussed above.

During manufacture of the vascular graft, the built-up region 686 may be formed during the same or a different processing step used to form the cuffed section 650. As integrally formed with the wall 660, the built-up region 686 is preferably formed from the same biocompatible material making up the cuffed portion 650. For example, in one exemplary embodiment, the cuffed portion 650 and the built-up region 686 may be formed of the same PTFE or FEP material. Of course, other biocompatible materials are contemplated.

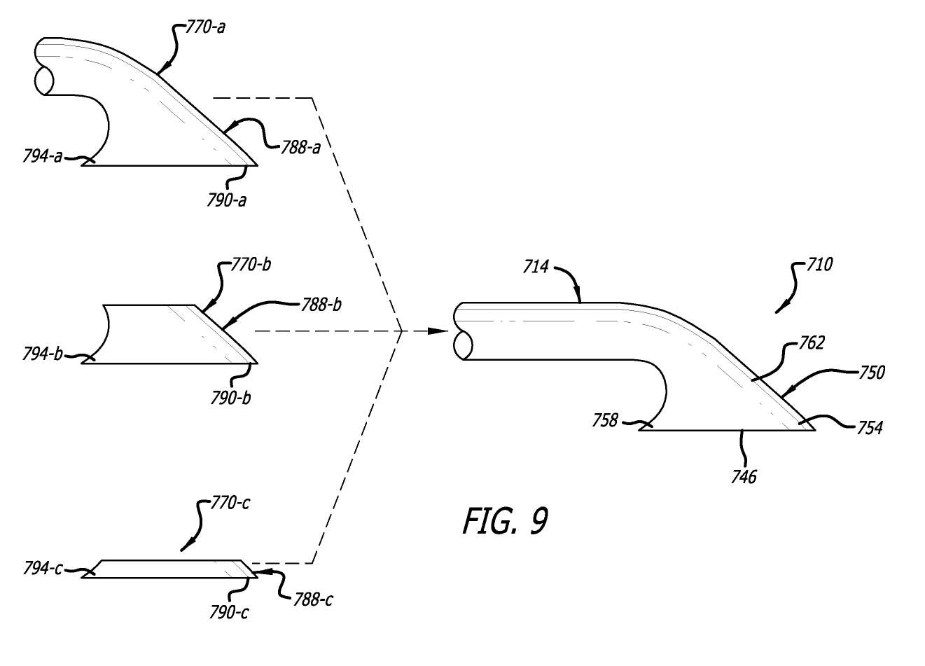

FIG. 9 illustrates a vascular graft prosthesis in accordance with still another exemplary embodiment of the present invention. The vascular graft 710 comprises a tubular member 714 defining a lumen, and an anastomotic component in the form of a cuffed section 750 extending from the tubular member 714 similar to that described above and shown in FIGS. 1, 2-A and 2-B, which description is incorporated herein. Like the vascular grafts discussed above, the vascular graft 710 of FIG. 9 further comprises means for reinforcing the anastomotic component to strengthen the margin 746 defined by a terminal end formation of the cuffed section 750. In this particular embodiment, means for reinforcing comprises various separate reinforcing components 770 adapted to fit over and secure to the cuffed section 750, which reinforcing components are shown as different types and configurations of skirts or sleeves, namely full-cuff sleeve 788-a, half-cuff sleeve 788-b and margin sleeve 788-c. As can be seen, each of the sleeves comprise a configuration and profile that matches, at least in part, the cuffed section 750 of the vascular graft 710. Although similar in geometry and configuration, they are sized sufficiently to fit over and secure (e.g., through sintering, staples, adhesives, etc.) to the exterior surface 762 of the cuffed section 750 prior to anastomosis. For example, full-cuff sleeve 788-a is shown as comprising a toe section 790-a and a heel section 794-a that each correspond to the toe and heel sections 754 and 758, respectively, of cuffed section 750. In addition, the full-cuff sleeve 788-a may comprise a tubular member extending therefrom for fitting over and securing to the tubular member 714. Depending on its configuration, the full-cuff sleeve 788-a may be fitted over the cuffed section 750 by inserting the tubular member 714 through the opening (not shown) in the full-cuff sleeve 788-a,and sliding the tubular member through the opening until the full-cuff sleeve 788-a is disposed about and fitted over the cuffed section 750, bringing the margins of the two components together, preferably within the same plane. Alternatively, a slit may be made in the full-cuff sleeve 788-a allowing it to be spread apart to facilitate its proper disposal about and fitting to the cuffed section 750 without having to feed the tubular member 714 through the opening in the full-cuff sleeve. Once in place and secured to the cuffed section 750, the full-cuff sleeve 788-a effectively becomes part of the vascular graft, increasing the wall thickness of the cuffed section 750, and ultimately strengthening and reinforcing the margin 746 for purposes as discussed herein.

FIG. 9 further illustrates half-cuff sleeve 788-b having toe and heel sections 790-b and 794-b, respectively, that also correspond to the toe and heel sections 754 and 758, respectively, of the cuffed portion 750. FIG. 9 still further illustrates margin-sleeve 788-c having toe and heel sections 790-c and 794-c, respectively, that also correspond to the toe and heel sections 754 and 758, respectively, of the cuffed portion 750. As it is typically only the margin that will need reinforcement, half-cuff sleeve 788-b and margin sleeve 788-c offer a more low profile alternative to the full-cuff sleeve 788-a described above. However, both the half-cuff sleeve and the margin sleeve are each intended to function in a similar manner, namely to strengthen the margin 746 of the cuffed section 750 of the vascular graft 710 for purposes as discussed herein.

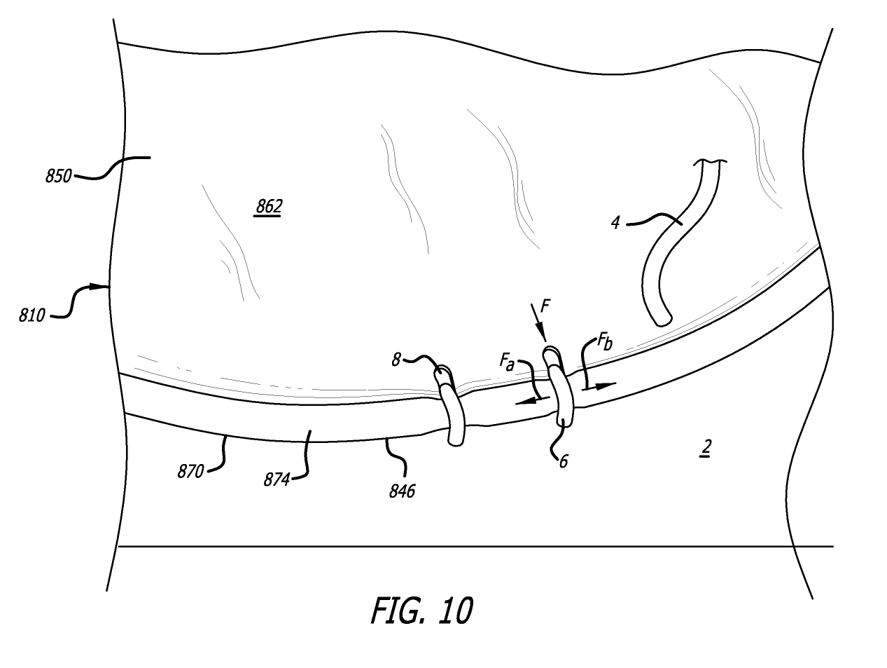

With reference to FIG. 10, illustrated is a partial view of an exemplary venous anastomotic site, wherein the venous end of an exemplary vascular graft prosthesis 810 is being anastomosed to a vein 2 at the venotomy site (this figure may also be considered to represent the vascular graft being anastomosed to an artery at an arteriotomy site). In this particular example, illustrated is a representation of an early stage suturing procedure, wherein two initial sutures 6 have been placed or sewn into the vascular graft 810 and vein 2 to initialize the anastomosis.

The vascular graft 810 comprises a cuffed section 850 having a reinforcement component 870 in the form of flex small beading 874 disposed about the margin 846 of the cuffed section 850 in a similar manner as discussed above.

During anastomosis, the suture line 4 is inserted through the cuffed section 850 of the vascular graft 810, drawn around the reinforcing beading 874, and then inserted through the wall of the vein 2 to create a suture 6 (having a loop), with the margin 846 and the beading 874 of the cuffed section 850 situated securely within the suture 6, thus securing the vascular graft 810 to the vein 2. As the sutures 6 are formed and sewn into place about the beading 874 to create a series of loops, the sutures 6 are subsequently tightened by the surgeon pulling taut the suture line 4, which action causes the sutures 6 to constrict or tighten around the reinforcing beading 874 of the vascular graft 810. As the suture line 4 is pulled taut, significant tensile forces are induced within the suture line 4 and the sutures 6, as indicated by the force arrow F. In addition, as the surgeon manipulates the vessel and/or vascular graft to obtain the desired positioning for continuing the suturing procedure, additional tensile forces can act between the vascular graft 810 and the vein 2. The combination of these generated tensile forces can induce one or both of suture line tearing and/or suture hole elongation. However, unlike prior related vascular grafts that suffer from suture line tearing and/or suture hole elongation, the present invention vascular graft 810, with its means for reinforcing, eliminates or at least considerably reduces the likelihood of suture line tearing. In addition, means for reinforcing functions to prevent and/or arrest or minimize suture hole elongation.

Slight suture hole elongation is shown in FIG. 10 for illustrative purposes to better explain the capabilities of the reinforcing means. Suture hole 8 is shown as being formed within the cuffed section 850 of the vascular graft 810 as a result of sewing suture line 4 and creating sutures 6. Suture hole 8 is further shown as being elongated a degree from its initial size and shape in the direction of the suture 6 as a result of various generated tensile forces F, as discussed above. However, further elongation of suture hole 8 is arrested, thus minimizing suture hole elongation, and suture line tearing prevented, as the suture line 4 is caused to interact with the reinforcing beading 874. The interaction of the suture line 4 and the suture 6 with the reinforcing beading 874 effectively functions to distribute the force F acting on the reinforcing beading 874 across a greater portion of the vascular graft 810. For example, as the suture 6 interacts with the beading 874 as a result of tensile forces F, tensile forces F are distributed, such as in a bi-directional manner, about the cuffed section 850 and the beading 874, as illustrated by resultant forces Fₐ and F_{b}. Resultant forces Fₐ and F_{b} obviously may be of different magnitude under certain conditions. By distributing the tensile forces across a greater portion of the vascular graft, adverse affects of the tensile forces are significantly diminished. Those skilled in the art will recognize that suture hole elongation can be further minimized by initially inserting the suture line 4 through the cuffed section 850 at a position or location closer to the margin 846, causing the suture 4 to initially be located more adjacent or juxtaposed to the reinforcing beading 874.

In short, it is believed that by reinforcing the anastomotic component of the vascular graft prosthesis, and therefore enhancing the anastomotic site, it is possible to reduce the risk of suture hole bleeding.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

More specifically, while illustrative exemplary embodiments of the invention have been described herein, the present invention is not limited to these embodiments, but includes any and all embodiments having modifications, omissions, combinations (e.g., of aspects across various embodiments), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given above.

What is claimed is:

1. A vascular graft prosthesis, comprising:
a generally tubular member defining a lumen for the passage of blood;
an anastomotic component connected to a distal end of the tubular member, the anastomotic component comprising a cuff having a terminal end formation defining a margin; and
a reinforcing element positioned proximate the margin, the reinforcing element defining a boundary of the margin.

2. The vascular graft prosthesis according to claim 1, wherein the reinforcing element distributes forces acting thereon laterally in a bi-directional manner about the cuff.

3. The vascular graft prosthesis according to claim 1, wherein the reinforcing element comprises a separate reinforcing component formed of a different biocompatible material than a biocompatible material of the cuff.

4. The vascular graft prosthesis according to claim 3, wherein the reinforcing component comprises flex beading selectively disposed about and secured to an outer surface of the terminal end formation.

5. The vascular graft prosthesis according to claim 4, wherein the flex beading is formed from a solid polytetrafluoroethylene material.

6. The vascular graft prosthesis according to claim 1, wherein the reinforcing element comprises a rib component having a generally rectangular cross-sectional shape formed of a different biocompatible material than a biocompatible material of the cuff.

7. The vascular graft prosthesis according to claim 1, wherein the reinforcing element extends both outwardly from an outer surface of the terminal end formation and inwardly from an inner surface of the terminal end formation.

8. The vascular graft prosthesis according to claim 1, further comprising a sleeve secured to the cuff, the sleeve including the reinforcing element.

9. The vascular graft prosthesis according to claim 8, wherein the sleeve is formed from expanded polytetrafluoroethylene and has a shape substantially similar to the cuff.

10. The vascular graft prosthesis according to claim 1, wherein the reinforcing element extends in a continuous, uninterrupted manner about an outer surface of the terminal end formation to define the entire boundary of the margin.

11. The vascular graft prosthesis according to claim 1, wherein the cuff and the reinforcing element are formed from the same biocompatible material.

12. The vascular graft prosthesis according to claim 1, wherein the reinforcing element is formed from a biocompatible material selected from the group consisting essentially of polytetrafluoroethylene (PTFE), expanded polytetrafluoroethylene (ePTFE), polyester, polyurethane, or fluoropolymers, such as perfluoroelastomers, and combinations thereof.

13. The vascular graft prosthesis according to claim 1, wherein the reinforcing element further comprises a bioactive agent.

14. The vascular graft prosthesis according to claim 1, wherein the reinforcing element comprises an increased wall thickness of the terminal end formation.

15. The vascular graft prosthesis according to claim 14, wherein the increased wall thickness is integrally formed with the terminal end formation.

16. The vascular graft prosthesis according to claim 1, wherein the reinforcing element minimizes suture hole elongation and prevents suture line tearing of the terminal end formation during anastomosis of the vascular graft prosthesis to a blood vessel.

17. A method for reinforcing a vascular graft prosthesis to enhance an anastomotic site, the vascular graft prosthesis including a generally tubular member defining a lumen for the passage of blood and an anastomotic component connected to a distal end of the tubular member, the anastomotic component comprising a cuff having a terminal end formation defining a margin, the method comprising reinforcing the margin along a substantial portion thereof.

18. The method according to claim 17, wherein the reinforcing step comprises depositing and securing a reinforcing component to a surface of the cuff, the reinforcing component comprising a different material than the material forming the cuff.

19. The method according to claim 17, wherein the reinforcing step comprises depositing and securing a solid polytetrafluoroethylene beading to an outer surface of the terminal end formation, the beading defining a boundary of the margin.

20. The method according to claim 17, wherein the reinforcing step comprises integrally forming a build-up material region proximate the margin, the build-up material region being formed from the same material as the material forming the cuff.
